(19) United States
(12) Patent Application Publication
(10) Pub. No.: US 2019/0041854 A1
(43) Pub. Date: Feb. 7, 2019

(54) SYSTEMS AND METHODS FOR PROVIDING EMERGENCY MEDICAL ASSISTANCE USING AN AUTOMATED ROBOTIC VEHICLE

(71) Applicant: Walmart Apollo, LLC, Bentonville, MA (US)

(72) Inventor: Andrew Millhouse, Gilbert, AZ (US)

(21) Appl. No.: 16/052,943

(22) Filed: Aug. 2, 2018

Related U.S. Application Data
(60) Provisional application No. 62/540,349, filed on Aug. 2, 2017.

(51) Int. Cl. G05D 1/00; G05D 1/02; G01C 21/20; G16H 10/60; H04W 4/02; H04W 4/90; A61B 5/11; A61F 17/00

(52) U.S. Cl. CPC G05D 1/0088; G05D 1/0234; G05D 1/0268; G05D 1/0246; G01C 21/206; A61B 2503/20; H04W 4/02; H04W 4/90; A61B 5/1117; A61F 17/00; G05D 2201/0206; G16H 10/60

(57) ABSTRACT

A system for providing emergency medical assistance using an automated robotic vehicle in a facility is provided. A mobile application executable on a mobile computing device uses at least one sensor in the mobile device to identify an incident occurrence, and transmits incident information to an incident computing device. The incident computing device receives the incident information from the mobile application and transmits a location of the incident to the automated robotic vehicle. The automated robotic vehicle travels to the location of the incident to provide emergency medical supplies for the incident to the individual.

### CROSS-REFERENCE TO RELATED PATENT APPLICATIONS

This application claims priority to U.S. Provisional Application No. 62/540,349, filed on Aug. 2, 2017, the content of which is hereby incorporated by reference in its entirety.

### BACKGROUND

Automated robotic vehicles (ARVs) are able to autonomously operate within a large physical facility such as a warehouse or distribution center. Once provided with a destination location, the ARVs can navigate through the facility using stored data and onboard sensors. Although able to operate autonomously, the ARVs may be in wireless network communication with a remote computing system during operation.

### BRIEF DESCRIPTION OF DRAWINGS

To assist those of skill in the art in making and using a system for providing emergency medical assistance and associated methods, reference is made to the accompanying figures. The accompanying figures, which are incorporated in and constitute a part of this specification, illustrate one or more embodiments of the invention and, together with the description, help to explain the invention. Illustrative embodiments are shown by way of example in the accompanying drawings and should not be considered as limiting. In the figures:

FIG. 1 illustrates an exemplary network environment suitable for a system providing emergency medical assistance using an automated robotic vehicle in a facility, in accordance with an exemplary embodiment;

FIG. 2 is an exemplary system for providing emergency medical assistance using an automated robotic vehicle in a facility, in accordance with an exemplary embodiment;

FIG. 3 is a block diagram of an exemplary computing device suitable for use in an exemplary embodiment;

FIG. 4 illustrates an exemplary method for providing emergency medical assistance using an automated robotic vehicle in a facility; and

FIG. 5 illustrates an automated robotic vehicle, in accordance with an exemplary embodiment.

### DETAILED DESCRIPTION

Described in detail herein are methods and systems for providing emergency medical assistance using an automated robotic vehicle in a facility. In an exemplary embodiment, the facility is a warehouse or a distribution center. The system includes a mobile application executable on a mobile computing device associated with an individual, such as an employee or a customer. The mobile application uses at least one sensor in the mobile device to identify an incident occurrence. For example, the incident occurrence may be a fall, a collision, or a medical situation, or some combination thereof, involving the individual. The mobile application further automatically transmits incident information to an incident computing device. The incident information includes a location of the incident and an incident type that classifies the incident. The incident computing device is in wireless communication with the mobile computing device and an automated robotic vehicle. The incident computing device is further configured to transmit the location of the incident occurrence to the automated robotic vehicle. Upon receiving the location from the incident computing device, the automated robotic vehicle travels to the location of the incident occurrence to provide emergency medical supplies to the individual.

In the exemplary embodiment, the automated robotic vehicle is a mobile robot that includes one or more bins containing emergency medical supplies. The automated robotic vehicle may be, but is not limited to, a drone, an unmanned ground vehicle, an unmanned aerial vehicle, an autonomous guided vehicle or an autonomous cart.

In some embodiments, the incident computing device can be located on or within the automated guided vehicle.

The system may improve medical service by minimizing an individual's wait time to receive emergency medical supplies by deploying the automated robotic vehicle, while also improving the ease and accuracy of providing medical services.

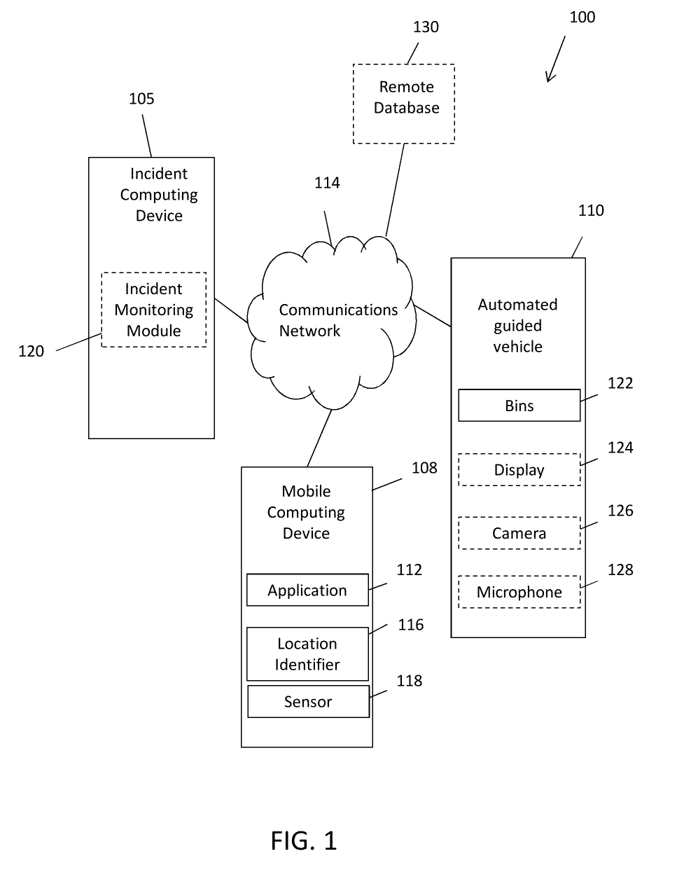

FIG. 1 illustrates an exemplary network environment suitable for a system 100 for providing emergency medical assistance using an automated robotic vehicle in a facility, in accordance with an exemplary embodiment. System 100 includes at least one incident computing device 105, at least one mobile computing device 108, and at least one automated robotic vehicle 110. As a non-limiting example, system 100 is associated with a physical facility such as a warehouse, distribution center, or retail store. In the exemplary embodiment, the incident computing device 105, the mobile computing device 108, and the automated robotic vehicle 110 are located within the physical facility.

In an exemplary embodiment, the mobile computing device 108 is a smartphone, tablet, or other handheld computing device, used by an employee or a customer. The mobile computing device 108 includes a mobile application 112 installed on the mobile computing device 108. The mobile application 112 is configured to communicate with the incident computing device 105 via a communications network 114. The mobile computing device 108 is able to generate a location identifier 116, such as a location determined via GPS Wi-Fi geolocation, or other location-based protocol. The mobile computing device includes at least one sensor 118 to identify an incident occurrence. In an exemplary embodiment, the sensor 118 may be a shock sensor for identifying a shock registering over a predefined g-force or an audio sensor for identifying a noise registering over a predefined decibel. The mobile application 112 may also generate a user interface enabling the individual operating the mobile device to report an incident such as a medical emergency. In one embodiment, the mobile application may be configured to track health data of the individual operating the mobile device and may detect the occurrence of a medical incident such as an unwanted change in heart rate, blood pressure or blood sugar level. For example, the mobile application may be in wireless communication with a medical bracelet configured to monitor a pulse rate or may make use of built in IR or RF capability on the mobile device to monitor other health conditions such as respiratory rates and heart function. The detection of health conditions may occur manually or automatically. The mobile application 112 is configured to automatically transmit incident information to the incident computing device 105 that includes a location of the incident occurrence and the type of incident.

In some embodiments, the incident computing device 105 may include an incident monitoring module 120 that includes one or more computer-executable processes that is dedicated to receive the incident information from the mobile application 112 and transmit the location of the incident occurrence to the automated robotic vehicle 110. The automated robotic vehicle 110 includes one or more bins 122 containing emergency medical supplies. In additional embodiments, the automated robotic vehicle 110 may also include a display 124 configured to display information to personnel responding to the individual based on the incident type. For example, in some embodiments, the automated robotic vehicle 110 is configured to receive an identification of the incident type from the incident computing device 105 and display medical instructions to personnel responding to the individual based on the incident type. In another embodiment, the display of information may include instructions for the individual suffering the incident so that the individual can access needed treatment via supplies carried by the automated robotic vehicle 110

In further embodiments, the automated robotic vehicle 110 includes at least one of a camera 126 and a microphone 128, and provides a transceiver or other two way communication capability for communicating with a third party using the camera 126 and/or the microphone 128. The communication with the third party may be provided via the incident computing device 105 or directly to the third party.

In some embodiments, the bins 122 are locked and organized based on incident types. In such an embodiment, the automated robotic vehicle 110 is further configured to receive an identification of the incident type from the incident computing device 105 and automatically unlock one or more bins of the bins 122 based on the incident type upon arriving at the location of the incident. As a non-limiting example, the automated robotic vehicle may have one bin that includes supplies used to treat diabetics, such as insulin and needles and may have a separate second bin that is used to treat cardiac situations such as nitroglycerin pills or cardiac stimulation devices. It will be appreciated that in other embodiments the automated robotic vehicle 110 may include only a single bin. In a further embodiment, a responder dispatched to the incident location may meet the automated robotic vehicle and be required to provide a code or biometric input prior to the bin being unlocked.

The communications network 114 can be any network over which information can be transmitted between devices communicatively coupled to the network. For example, one or more portions of communications network 114 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

In some embodiments, the automated robotic vehicle 110 is configured to receive an identifier (e.g., a social security number, a medical identifier, a patient identifier, etc.) of the individual to whom the automated robotic vehicle is responding. For example, a responder may enter the identifier for an individual suffering an injury into the automated robotic vehicle 110 using the display 124 and a keypad associated with the automated robotic vehicle 110. The automated robotic vehicle 110 transmits the identifier to the incident computing device 105 via communications network 114. The incident computing device 105 retrieves medical records associated with the identifier from a remote database 130, such as a hospital database or a medical records repository. The incident computing device 105 transmits the medical records to the automated robotic vehicle 110. The automated robotic vehicle 110 displays the medical records on the display 124, enabling, for example, the responder to view the medical records of the individual.

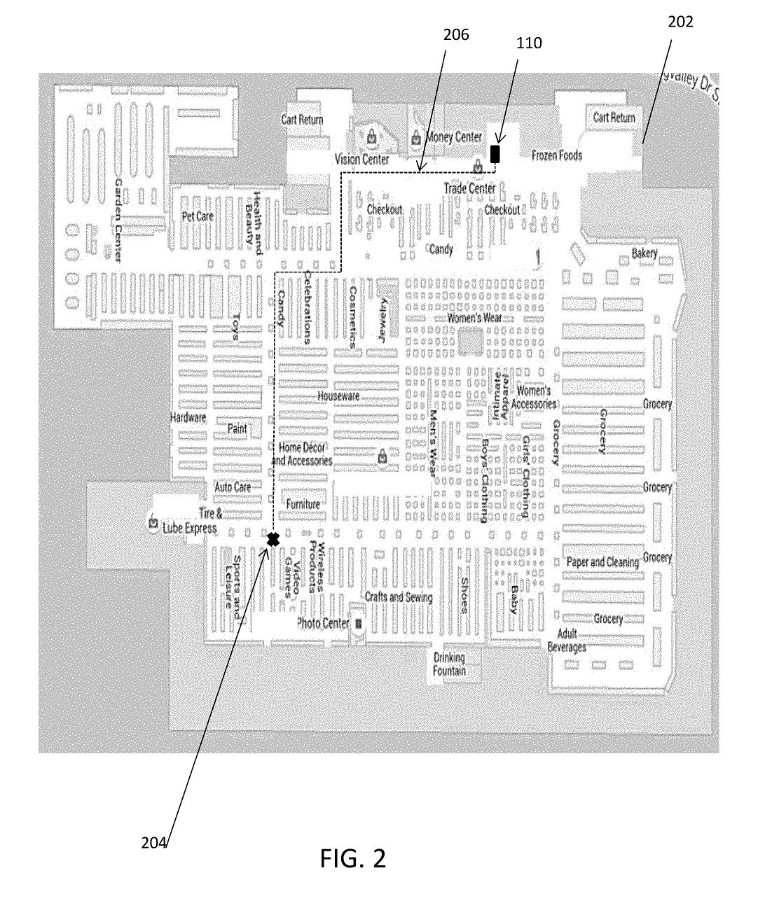

FIG. 2 is a store diagram 200 illustrating the exemplary system for providing emergency medical assistance using an automated robotic vehicle 110 in a facility 202, in accordance with an exemplary embodiment. A mobile application 112 executable on a mobile computing device 108 associated with an individual is located within the facility 202. The mobile application 112 uses at least one sensor in the mobile computing device 108 to identify an incident occurrence. As non-limiting examples, the incident occurrence includes at least one of a fall, a collision, and a medical incident. The sensor 118 includes at least one of a shock sensor for identifying a shock registering over a predefined g-force or an audio sensor for identifying a noise registering over a predefined decibel. As noted above, the mobile application 112 may also provide a user interface to self-report an incident and may include health tracking to detect a medical condition.

Upon identifying an incident occurrence, the mobile application 112 automatically transmits incident information to an incident computing device 105. The incident information includes a location of the incident occurrence and an incident type. FIG. 2 identifies an exemplary location 204 of an incident occurrence. The incident type is at least one of a fall, a collision, and a medical incident depending on the incident occurrence. The incident computing device 105 is in wireless communication with the mobile application 112 executing on the mobile computing device 108 and the automated robotic vehicle 110. The incident computing device 105 is configured to receive the incident information from the mobile application 112 and transmit the location 204 of the incident occurrence to the automated robotic vehicle 110 located within the facility 202. The automated robotic vehicle 110 includes one or more bins containing emergency medical supplies. The automated robotic vehicle 110 travels to the location 204 of the incident occurrence to provide emergency medical supplies for the incident occurrence to the individual. Depending on the type of incident, the incident computing device 105 may also send further instructions to the automated robotic vehicle regarding what to display on a display screen and what bins to unlock or prepare to unlock (once provided with authorized input).

In one embodiment, the automated robotic vehicle 110 uses floor-based markers for traveling to the location 204 of the incident occurrence. For example, in one embodiment, the automated robotic vehicle 110 follows markers or tape on the floor of the facility 202. The tape for the guide path may be one of two styles: magnetic or colored. The automated robotic vehicle 110 is fitted with the appropriate guide sensors to follow the path of the tape. A flexible magnetic bar can also be embedded in the floor wire but works under the same provision as magnetic tape. Alternatively, the automated robotic vehicle may follow projections on the floor from facility sensors. In an alternative embodiment, the automated robotic vehicle 110 uses onboard location sensors for traveling to the location 204 of the incident occurrence. For example, the automated robotic vehicle may use inertial navigation. With inertial guidance, transponders are embedded in the floor of the facility 202. The automated robotic vehicle uses these transponders to verify that the vehicle is on course. Inertial navigation can include use of magnets embedded in the floor of the facility 202 that the automated robotic vehicle 110 can read and follow.

In further embodiments, the automated robotic vehicle 110 uses vision guidance for navigation. Vision-Guided automated robotic vehicle 110 operate by using cameras to record features along the route, allowing the automated robotic vehicle 110 to replay the route by using the recorded features to navigate. The automated robotic vehicle 110 can use geo-guidance technology to detect and identify, for example, columns, racks and walls within the facility, and use these fixed references to position itself and determine its route.

In additional embodiments, the automated robotic vehicle 110 uses lasers for navigation. The automated robotic vehicle 110 carries a laser transmitter and receiver on a rotating turret. The laser is transmitted and received by the same sensor. The angle and (sometimes) distance to any reflectors that in line of sight and in range are automatically calculated. This information is compared to the map of the reflector layout stored in a memory of the automated robotic vehicle 110. This allows the navigation system to triangulate the current position of the automated robotic vehicle 110. The current position is compared to the path programmed into the reflector layout map. The steering is adjusted accordingly to keep the automated robotic vehicle 110 on track.

It will be appreciated that other means of navigation other than those discussed herein may also be employed by the automated robotic vehicle without departing from the scope of the present invention.

Traffic control for the automated robotic vehicle 110 can be carried out locally or by software running on a fixed computer elsewhere in the facility. Local methods include zone control, forward sensing control, and combination control. For example, forward sensing control uses collision avoidance sensors to avoid the automated robotic vehicle 110 colliding with objects and customers in the area.

In additional embodiments, the incident computing device 105 is further configured to notify pre-determined types of personnel based on the location of the incident and the incident type. The personnel may be at least one of emergency medical personnel, one or more facility managers, or one or more co-workers of the individual such as an incident response team. For example, the personnel may be notified via a paging system within the facility.

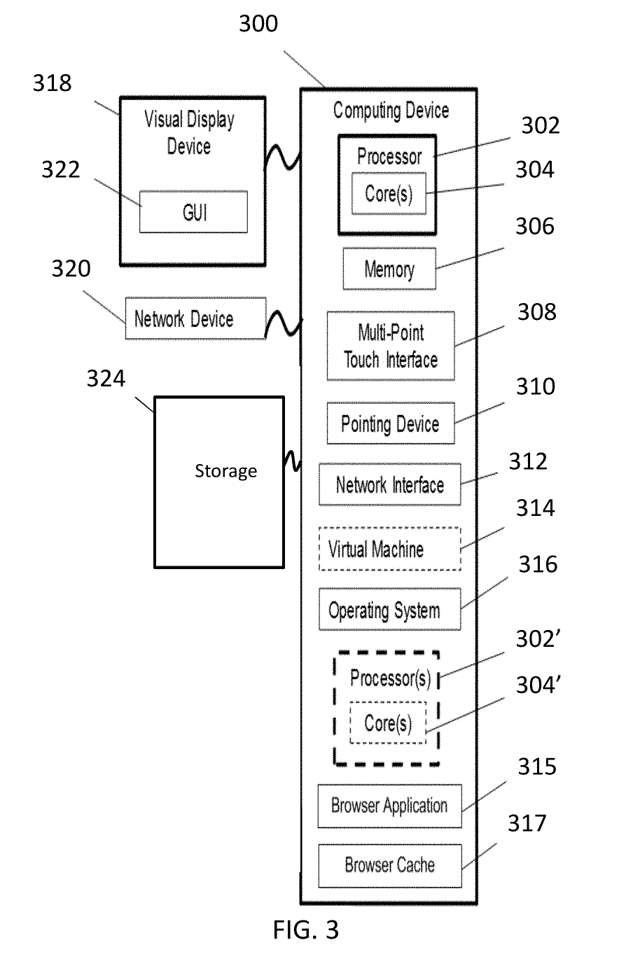

FIG. 3 is a block diagram of an example computing device 300 that can be used to perform one or more steps of the methods provided by exemplary embodiments. In an exemplary embodiment, computing device 300 is an incident computing device 105 as shown in FIG. 1 and/or a mobile computing device 108 shown in FIG. 1. Computing device 300 includes one or more non-transitory computer-readable media for storing one or more computer-executable instructions or software for implementing exemplary embodiments described herein. The non-transitory computer-readable media can include, but are not limited to, one or more types of hardware memory, non-transitory tangible media (for example, one or more magnetic storage disks, one or more optical disks, one or more USB flashdrives), and the like. For example, a memory 306 included in computing device 300 can store computer-readable and computer-executable instructions or software for implementing exemplary embodiments described herein. Computing device 300 also includes a processor 302 and an associated core 304, and optionally, one or more additional processor(s) 302′ and associated core(s) 304′ (for example, in the case of computer systems having multiple processors/cores), for executing computer-readable and computer-executable instructions or software stored in memory 306 and other programs for controlling system hardware. Processor 302 and processor(s) 302′ can each be a single core processor or multiple core (304 and 304′) processor. Computing device 300 may also include a browser application 315 and a browser cache 317 to enable a user to information on computing device 300.

Virtualization can be employed in computing device 300 so that infrastructure and resources in the computing device can be shared dynamically. A virtual machine 314 can be provided to handle a process running on multiple processors so that the process appears to be using only one computing resource rather than multiple computing resources. Multiple virtual machines can also be used with one processor.

Memory 306 can include a computer system memory or random access memory, such as DRAM, SRAM, EDO RAM, and the like. Memory 306 can include other types of memory as well, or combinations thereof. In some embodiments, a customer can interact with computing device 300 through a graphical user interface (GUI) 322 associated with a visual display device 318, such as a touch screen display or computer monitor. Visual display device 318 may also display other aspects, elements and/or information or data associated with exemplary embodiments. Computing device 300 may include other I/O devices for receiving input from a customer, for example, a keyboard or any suitable multi-point touch interface 308, a pointing device 310 (e.g., a pen, stylus, mouse, or trackpad). The multi-point touch interface 308 and pointing device 310 may be coupled to visual display device 318. Computing device 300 may include other suitable conventional I/O peripherals.

Computing device 300 can also include one or more storage devices 324, such as a hard-drive, CD-ROM, or other computer readable media, for storing data and computer-readable instructions and/or software, that implements embodiments of the system, as described herein, or portions thereof. Exemplary storage device 324 can also store one or more storage devices for storing any suitable information required to implement exemplary embodiments.

Computing device 300 can include a network interface 312 configured to interface via one or more network devices 320 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 312 can include a built-in network adapter, network interface card, PCMCIA network card, card bus network adapter, wireless network adapter, USB network adapter, modem or any other device suitable for interfacing computing device 300 to any type of network capable of communication and performing the operations described herein. Moreover, computing device 300 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

Computing device 300 can run any operating system 316, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 316 can be run in native mode or emulated mode. In an exemplary embodiment, the operating system 316 can be run on one or more cloud machine instances.

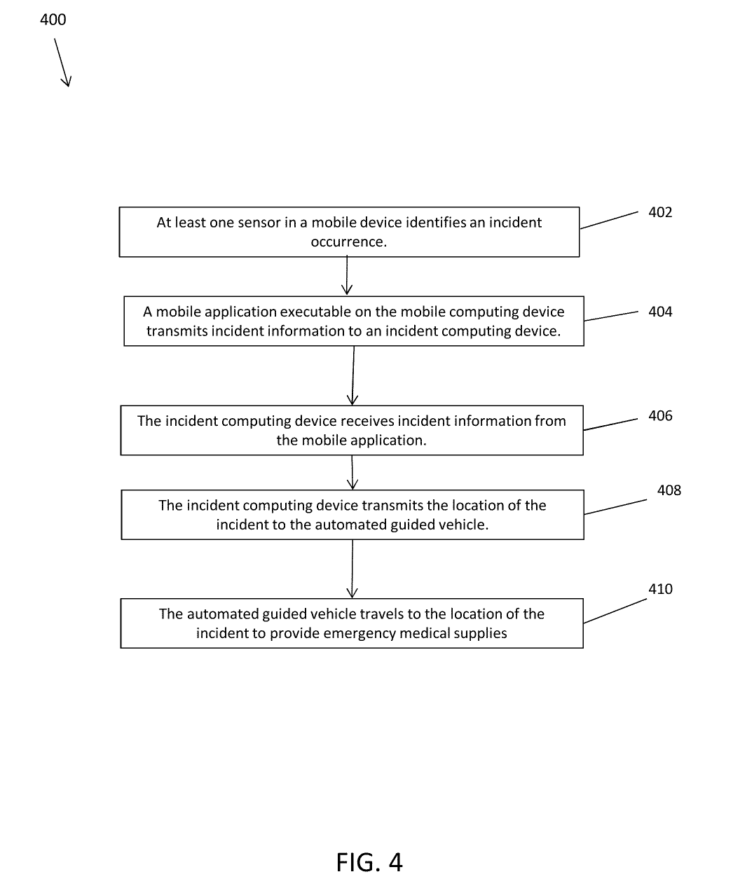

FIG. 4 illustrates an exemplary method 400 for providing emergency medical assistance using an automated robotic vehicle 110 in a facility, according to an exemplary embodiment. At step 402, at least one sensor in a mobile computing device 108 identifies an incident occurrence. At step 404, a mobile application executable on the mobile computing device 108 transmits the incident information to an incident computing device 105. The incident information includes at least a location of the incident occurrence and an incident type. At step 406, the incident computing device 105 receives the incident information from the mobile application. At step 408, the incident computing device 105 transmits the location of the incident occurrence to the automated robotic vehicle 110. At step 410, the automated robotic vehicle 110 travels to the location of the incident occurrence to provide emergency medical supplies for the incident occurrence to an individual. The automated robotic vehicle 110 includes one or more bins containing emergency medical supplies.

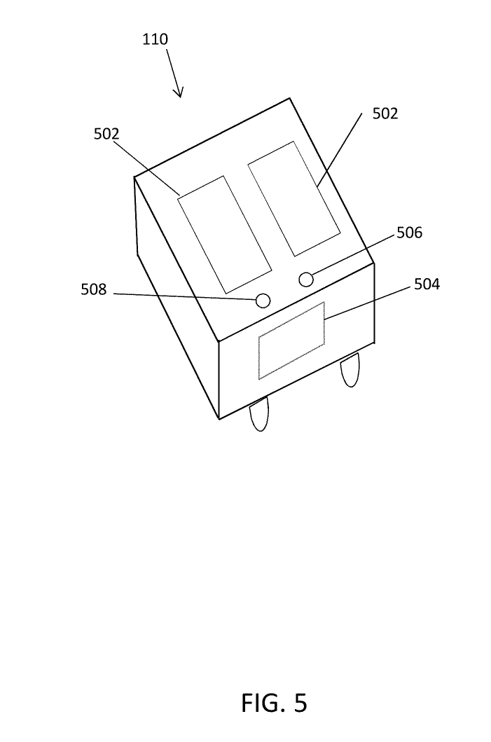

FIG. 5 illustrates an automated robotic vehicle 110, in accordance with an exemplary embodiment. The automated robotic vehicle 110 is a mobile robot that includes one or more bins 502 containing emergency medical supplies. As discussed herein, bins 502 may include locking mechanisms. While FIG. 5 illustrates the automated robotic vehicle 110 with two bins 502, in additional embodiments, the automated robotic vehicle 110 can include a greater or lesser number of bins 502. In additional embodiments, the automated robotic vehicle 110 may include a display 504, a camera 506, and/or a microphone 508, as described herein. Automated robotic vehicle 110 further includes any mechanisms, circuitry, processor(s), sensor(s), and non-transitory computer-readable media for storing one or more computer-executable instructions or software for implementing exemplary embodiments as described herein.

The description herein is presented to enable any person skilled in the art to create and use a computer system configuration and related method and systems for improving access to electronic data. Various modifications to the example embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention may be practiced without the use of these specific details. In other instances, well-known structures and processes are shown in block diagram form in order not to obscure the description of the invention with unnecessary detail. Thus, the present disclosure is not intended to be limited to the embodiments shown, but is to be accorded the widest scope consistent with the principles and features disclosed herein.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes multiple system elements, device components or method steps, those elements, components or steps can be replaced with a single element, component or step. Likewise, a single element, component or step can be replaced with multiple elements, components or steps that serve the same purpose. Moreover, while exemplary embodiments have been shown and described with references to particular embodiments thereof, those of ordinary skill in the art will understand that various substitutions and alterations in form and detail can be made therein without departing from the scope of the invention. Further still, other aspects, functions and advantages are also within the scope of the invention.

Portions or all of the embodiments of the present invention may be provided as one or more computer-readable programs or code embodied on or in one or more non-transitory mediums. The mediums may be, but are not limited to a hard disk, a compact disc, a digital versatile disc, a flash memory, a PROM, a RAM, a ROM, or a magnetic tape. In general, the computer-readable programs or code may be implemented in many computing languages.

Exemplary flowcharts are provided herein for illustrative purposes and are non-limiting examples of methods. One of ordinary skill in the art will recognize that exemplary methods can include more or fewer steps than those illustrated in the exemplary flowcharts, and that the steps in the exemplary flowcharts can be performed in a different order than the order shown in the illustrative flowcharts.

We claim:

1. A system for providing emergency medical assistance using an automated robotic vehicle in a facility, the system comprising:
a mobile application executable on a mobile computing device associated with an individual, the mobile application when executed:
uses at least one sensor in the mobile device to identify an incident occurrence, and
automatically transmits incident information to an incident computing device, wherein the incident information includes a location of the incident and an incident type;
an automated robotic vehicle that includes one or more bins containing emergency medical supplies; and
the incident computing device in wireless communication with the mobile computing device and the automated robotic vehicle, the incident computing device equipped with a processor and configured to:
receive incident information from the mobile application, the incident information including a location of an incident and an incident type, and
transmit the location of the incident to the automated robotic vehicle, whereupon the automated guided vehicle travels to the location of the incident to provide emergency medical supplies for the incident to the individual, the automated robotic vehicle using at least one of floor-based markers or onboard location sensors for traveling to the location of the incident.

2. The system of claim 1, wherein one or more bins are locked and organized based on incident types and the automated robotic vehicle is further configured to receive an identification of the incident type from the incident computing device and unlock one or more bins based on the incident type.

3. The system of claim 1, wherein the incident computing device is further configured to notify pre-determined types of personnel based on the location of the incident and the incident type, and wherein the personnel is at least one of emergency medical personnel, one or more facility managers, or one or more co-workers of the individual.

4. The system of claim 3, wherein the personnel is notified via a paging system within the facility.

5. The system of claim 1, wherein the incident type is at least one of a fall, a collision, and a medical incident.

6. The system of claim 1, wherein the automated robotic vehicle further includes:
a display, the display configured to display instructions to personnel responding to the individual based on the incident type.

7. The system of claim 1, the automated robotic vehicle further includes at least one of a camera and a microphone, wherein the automated robotic vehicle further includes two way communication capability for communicating with a third party using the at least one of the camera and the microphone.

8. The system of claim 1, wherein the at least one sensor includes at least one of a shock sensor for identifying a shock registering over a predefined g-force or an audio sensor for identifying a noise registering over a predefined decibels.

9. A method for providing emergency medical assistance using an automated robotic vehicle in a facility, the method comprising:
using at least one sensor in a mobile device to identify an incident occurrence;
transmitting, by a mobile application executable on the mobile computing device associated with an individual, automatically incident information to an incident computing device, wherein the incident information includes a location of the incident and an incident type;
receiving, by an incident computing device in wireless communication with the mobile computing device and an automated robotic vehicle that includes one or more bins containing emergency medical supplies, incident information from the mobile application, the incident information including a location of an incident and an incident type; and
transmitting, by the incident computing device, the location of the incident to the automated robotic vehicle;
traveling, by the automated robotic vehicle, to the location of the incident to provide emergency medical supplies for the incident to the individual, the automated robotic vehicle using at least one of floor-based markers or onboard location sensors for traveling to the location of the incident.

10. The method of claim 9, wherein the one or more bins are locked and organized based on incident types, the method further comprising:
receiving, by the automated robotic vehicle, an identification of the incident type from the incident computing device; and
unlocking one or more bins based on the incident type.

11. The method of claim 9, further comprising notifying, by the incident computing device, pre-determined types of personnel based on the location of the incident and the incident type, wherein the personnel is at least one of emergency medical personnel, one or more facility managers, or one or more co-workers of the individual.

12. The method of claim 11, further comprising notifying the personnel via a paging system within the facility.

13. The method of claim 9, wherein the incident type is at least one of a fall, a collision, and a medical incident.

14. The method of claim 9, wherein the automated robotic vehicle further includes a display, the method further comprising displaying on the display instructions to personnel responding to the individual based on the incident type.

15. The method of claim 9, wherein the automated robotic vehicle further includes two way communication capability, the method further comprising communicating with a third party using the automated robotic vehicle.

16. The method of claim 9, wherein the at least one sensor includes at least one of a shock sensor for identifying a shock registering over a predefined g-force or an audio sensor for identifying a noise registering over a predefined decibels.

17. A non-transitory computer-readable medium storing instructions for providing emergency medical assistance using an automated robotic vehicle in a facility, the instructions when executed causing at least one computing device to:
using at least one sensor in a mobile device to identify an incident occurrence;
transmitting, by a mobile application executable on the mobile computing device associated with an individual, automatically incident information to an incident computing device, wherein the incident information includes a location of the incident and an incident type;
receiving, by an incident computing device in wireless communication with the mobile computing device and an automated robotic vehicle that includes one or more bins containing emergency medical supplies, incident information from the mobile application, the incident information including a location of an incident and an incident type; and
transmitting, by the incident computing device, the location of the incident to the automated robotic vehicle;
traveling, by the automated robotic vehicle, to the location of the incident to provide emergency medical supplies for the incident to the individual, the automated robotic vehicle using at least one of floor-based markers or onboard location sensors for traveling to the location of the incident.

18. The non-transitory computer readable medium of 17, wherein one or more bins are locked and organized based on incident types, wherein the instructions when executed further cause the automated robotic vehicle to:
receive an identification of the incident type from the incident computing device; and
unlocking one or more bins based on the incident type.

19. The non-transitory computer readable medium of 17, wherein the instructions when executed further cause the incident computing device to notify pre-determined types of personnel based on the location of the incident and the incident type, wherein the personnel is at least one of emergency medical personnel, one or more facility managers, or one or more co-workers of the individual.

20. The method of claim 19, wherein the instructions when executed further notify the personnel via a paging system within the facility.

21. The non-transitory computer readable medium of 17, wherein the incident type is at least one of a fall, a collision, and a medical incident.

22. The non-transitory computer readable medium of 17, wherein the automated robotic vehicle further includes a display, wherein the instructions when executed further cause the display to display instructions to personnel responding to the individual based on the incident type.

23. The non-transitory computer readable medium of 17, wherein the automated robotic vehicle further includes two way communication capabilities for communicating with a third party using the automated robotic vehicle.

24. The non-transitory computer readable medium of 17, wherein the at least one sensor includes at least one of a shock sensor for identifying a shock registering over a predefined g-force or an audio sensor for identifying a noise registering over a predefined decibels.
